SEQUENCE LISTING (7 sequences)

SEQ ID NO: 1 - protein, 10 aa, Mus musculus
GYISSDQILN

SEQ ID NO: 2 - protein, 17 aa, Artificial Sequence
  Synthetic
  MISC_FEATURE (12)..(12): X is any amino acid
RIYPVTGVTQYXHKFVG

SEQ ID NO: 3 - protein, 6 aa, Artificial Sequences
  Synthetic
GETFDS

SEQ ID NO: 4 - protein, 17 aa, Artificial Sequence
  Synthetic
  MISC_FEATURE (8)..(8): X at position 8 is F, Y, or W
  MISC_FEATURE (15)..(15): X at position 15 is C, G, S, or T
KSNQNLLXSGNRRYXLV

SEQ ID NO: 5 - protein, 7 aa, Mus musculus
WASDRSF

SEQ ID NO: 6 - protein, 9 aa, Mus musculus
QQHLDIPYT

SEQ ID NO: 7 - protein, 15 aa, Artificial Sequence
  Synthetic
GGGGSGGGGSGGGGS

(19) United States
(12) Patent Application Publication
(10) Pub. No.: US 2018/0371089 A1
(43) Pub. Date: Dec. 27, 2018

(54) ASYMMETRIC HETERODIMERIC FC-SCFV FUSION ANTI-GLOBO H AND ANTI-CD3 BISPECIFIC ANTIBODY AND USES THEREOF IN CANER THERAPY

(71) Applicant: Development Center for Biotechnology, New Taipei City (TW)

(72) Inventors: Chia-Cheng WU, New Taipei City (TW); Tzu-Yin LIN, New Taipei City (TW); Yu-Jung CHEN, New Taipei City (TW); Chao-Yang HUANG, New Taipei City (TW)

(73) Assignee: Development Center for Biotechnology, New Taipei City (TW)

(21) Appl. No.: 16/016,525

(22) Filed: Jun. 22, 2018

Related U.S. Application Data
(60) Provisional application No. 62/523,280, filed on Jun. 22, 2017.

(51) Int. Cl. C07K 16/28

(52) U.S. Cl. CPC C07K 16/2809; A61P 35/00; C07K 2317/622; C07K 2317/55

(57) ABSTRACT

A bispecific anti-Globo H antibody includes an anti-Globo H antibody that binds specifically to Globo H; and a T-cell targeting domain fused to a CH3 domain of a heavy chain of the anti-Globo H antibody, wherein the T-cell targeting domain binds specifically to an antigen on T-cells; and wherein the anti-Globo H antibody comprises mutations at an effector binding site such that the bispecific anti-Globo H antibody has a diminished effector function. The T-cell targeting domain is a ScFv or Fab from an anti-CD3 antibody.

### BACKGROUND OF INVENTION

### Field of the Invention

The present invention relates to antibody engineering, particularly to asymmetric heterodimeric bispecific antibodies for cancer therapy.

### Background Art

Globo H is a hexasaccharide (Fuc-α1→2Gal-β1→3Gal-NAc-β1→3Gal-α1→4Gal-β1→4Glcβ1-) that is overexpressed on the surface of various epithelial cancer cells, including breast, colon, ovarian, pancreatic, lung, and prostate cancer cells. Therefore, Globo H is a promising diagnostic/therapeutic target.

Although antibodies against Globo H are useful, there remains a need for improved therapeutic agents using anti-Globo H antibodies.

### SUMMARY OF INVENTION

The present invention relates to bispecific anti-Globo H antibodies containing a T cell targeting (e.g., anti-CD3) domain and their uses in cancer therapy.

One aspect of the invention relates to bispecific anti-Globo H antibodies. A bispecific anti-Globo H antibody in accordance with one embodiment of the invention includes an anti-Globo H antibody that binds specifically to Globo H; and a T-cell targeting domain fused to a CH3 domain of a heavy chain of the anti-Globo H antibody, wherein the T-cell targeting domain binds specifically to an antigen on T-cells; and wherein the anti-Globo H antibody comprises mutations at an effector binding site such that the bispecific anti-Globo H antibody has a diminished effector function.

In accordance with some embodiments of the invention, the bispecific anti-Globo H antibodies may have T-cell targeting domain, which may be a ScFv or Fab, which may be derived from an anti-CD3 antibody.

In accordance with some embodiments of the invention, the mutations at the effector binding site may include L234A and L235A mutations or L235A and G237A mutations.

The bispecific anti-Globo H antibody may also include asymmetric heterodimeric heavy chains having knob-and-hole structures, which are generated by mutations in the CH3 domains. These mutations may include S354C and T366W for the knob arm, and Y349C, T366S, L368A, and Y407V for the hole arm.

Other aspect of the invention will become apparent with the following description.

### BRIEF DESCRIPTION OF THE DRAWINGS

FIG. 1 shows a schematic illustrating examples of asymmetric dimeric bispecific anti-Globo H antibodies containing ScFv anti-CD3 fusions in accordance with embodiments of the invention.

FIG. 2 shows the nucleotide sequences of a hole arm, with L234A, L235A, Y349C, T366S, L368A, and Y407V mutations, of an anti-Globo H antibody in accordance with embodiments of the invention.

FIG. 3 shows the nucleotide sequence of a hole arm, with L235A, G237A, Y349C, T366S, L368A, and Y407V mutations, of an anti-Globo H antibody in accordance with one embodiment of the invention.

FIG. 4 shows the nucleotide sequence of a knob arm, with L234A, L235A, S354C and T366W, mutations of an anti-Globo H antibody in accordance with one embodiment of the invention.

FIG. 5 shows the nucleotide sequence of a knob arm, with L235A, G237A, S354C and T366W mutations, of an anti-Globo H antibody in accordance with one embodiment of the invention.

FIG. 6 shows an amino acid sequence of a hole arm with, L234A, L235A, Y349C, T366S, L368A, and Y407V mutations, of an anti-Globo H antibody in accordance with one embodiment of the invention.

FIG. 7 shows an amino acid sequence of a knob arm, with L234A, L235A, S354C and T366W mutations, of an anti-Globo H antibody in accordance with one embodiment of the invention.

FIG. 8 shows an amino acid sequence of a hole arm, with L235A, G237A, Y349C, T366S, L368A, and Y407V mutations, of an anti-Globo H antibody in accordance with one embodiment of the invention.

FIG. 9 shows an amino acid sequence of a knob arm, with L235A, G237A, S354C and T366W mutations, of an anti-Globo H antibody in accordance with one embodiment of the invention.

FIG. 10 shows the nucleotide sequence of a linker in accordance with one embodiment of the invention.

FIG. 11 shows the nucleotide sequence of an anti-CD3 ScFv in accordance with one embodiment of the invention.

FIG. 12 shows the amino acid sequence of a linker in accordance with one embodiment of the invention.

FIG. 13 shows the amino acid sequence of an anti-CD3 ScFv in accordance with one embodiment of the invention.

FIG. 14 shows an anti-Globo H×anti-CD3 bispecific antibody effectively kills Globo H expressing breast cancer cell line HCC1428 in the presence of human PBMC in accordance with embodiments of the invention.

FIG. 15 shows an anti-Globo H×anti-CD3 bispecific antibody effectively kills Globo H expressing breast cancer cell line HCC1428 in the presence of human T cells in accordance with embodiments of the invention.

FIG. 16 shows Fc Mutation of L234A and L235A or L235A and G237A in the CH2 domain completely inhibited antibody-mediated complement-dependent cytotoxicity (CDC) in accordance with embodiments of the invention.

FIG. 17 shows non-specific T cell activation and IL-2 production induced by Fc-anti-CD3 ScFv fusion domain were completely diminished in L234A and L235A or L235A and G237A mutants in accordance with one embodiment of the invention.

FIG. 18 shows non-specific T cell activation and TNF-α production induced by Fc-anti-CD3 ScFv fusion domain were completely diminished L234A and L235A or L235A and G237A mutants in accordance with one embodiment of the invention.

FIG. 19 shows non-specific T cell activation and IFN-γ production induced by Fc-anti-CD3 ScFv fusion domain were completely diminished in L234A and L235A or L235A and G237A mutants in accordance with one embodiment of the invention.

FIG. 20 shows non-specific T cell activation and perforin production induced by Fc-anti-CD3 ScFv fusion domain were completely diminished in L234A and L235A or L235A and G237A mutants in accordance with one embodiment of the invention.

FIG. 21 shows non-specific T cell activation and granzyme A production induced by Fc-anti-CD3 ScFv fusion domain were completely diminished in L234A and L235A or L235A and G237A mutants in accordance with one embodiment of the invention.

FIG. 22 shows non-specific T cell activation and granzyme B production induced by Fc-anti-CD3 ScFv fusion domain were completely diminished in L234A and L235A or L235A and G237A mutants in accordance with one embodiment of the invention.

FIG. 23 shows AHFS anti-Globo H×anti-CD3 BsAb effectively activates T cell and induces IL-2 production in a tumor target cell-dependent manner in accordance with one embodiment of the invention.

FIG. 24 shows AHFS anti-Globo H×anti-CD3 BsAb effectively activates T cell and induces TNF-α production in a tumor target cell-dependent manner in accordance with one embodiment of the invention.

FIG. 25 shows AHFS anti-Globo H×anti-CD3 BsAb effectively activates T cell and induces IFN-γ production in a tumor target cell-dependent manner in accordance with one embodiment of the invention.

FIG. 26 shows AHFS anti-Globo H×anti-CD3 BsAb effectively activates T cell and induces perforin production in a tumor target cell-dependent manner in accordance with one embodiment of the invention.

FIG. 27 shows AHFS anti-Globo H×anti-CD3 BsAb effectively activates T cell and induces granzyme A production in a tumor target cell-dependent manner in accordance with one embodiment of the invention.

FIG. 28 shows AHFS anti-Globo H×anti-CD3 BsAb effectively activates T cell and induces granzyme B production in a tumor target cell-dependent manner in accordance with one embodiment of the invention.

### DETAILED DESCRIPTION

Embodiments of the invention relate to bispecific asymmetric antibodies against Globo H and uses of these antibodies in the treatment of cancers. An asymmetric antibody contains two heavy chains that are not identical. One of the heavy chain functions as a knob arm, and the other heavy chain functions as a hole arm that can accommodate the knob. The knob and hole structures may be engineered (e.g., by site-directed mutagenesis) in the third constant domain of the heavy chains, CH3. The complementarity of the knob and hole facilitates the formation of asymmetric antibodies. (A. M. Merchant et al., “An efficient route to human bispecific IgG,” Nat. Biotechnol., 1998, 16:677-81; doi: 10.1038/nbt0798-677).

The asymmetric heterodimeric bispecific antibodies of the invention contain variable domains that bind specifically to Globo H. In addition, these antibodies each contain an ScFv or Fab fragment of an antibody that targets an antigen on T-cells (e.g., an anti-CD3). Therefore, antibodies of the invention are bispecific antibodies—i.e., one domain specifically binds Glob H and the other domain specifically binds a T-cell antigen (e.g., CD3). By having a binding domain (e.g., a ScFv or Fab fragment) that specifically target T-cells, these antibodies are endowed with T-cell recruiting abilities to facilitate T-cell mediated cytotoxicity. Because these antibodies bind specifically to Globo H, one can ensure that the T-cell mediated cytotoxicity is directed toward cells that express Globo H antigens, such as cancers of epithelial origins.

Furthermore, in order to ensure the T-cell cytotoxicity is specifically directed towards the Globo H-expressing cells, the effector functions of these antibodies may be silenced or reduced. While antibody effector functions, such as antibody-dependent cellular cytotoxicity (ADCC) and complement-dependent cytotoxicity (CDC), are desirable in immune therapies, these effector functions are not desirable with the bispecific antibodies of the invention.

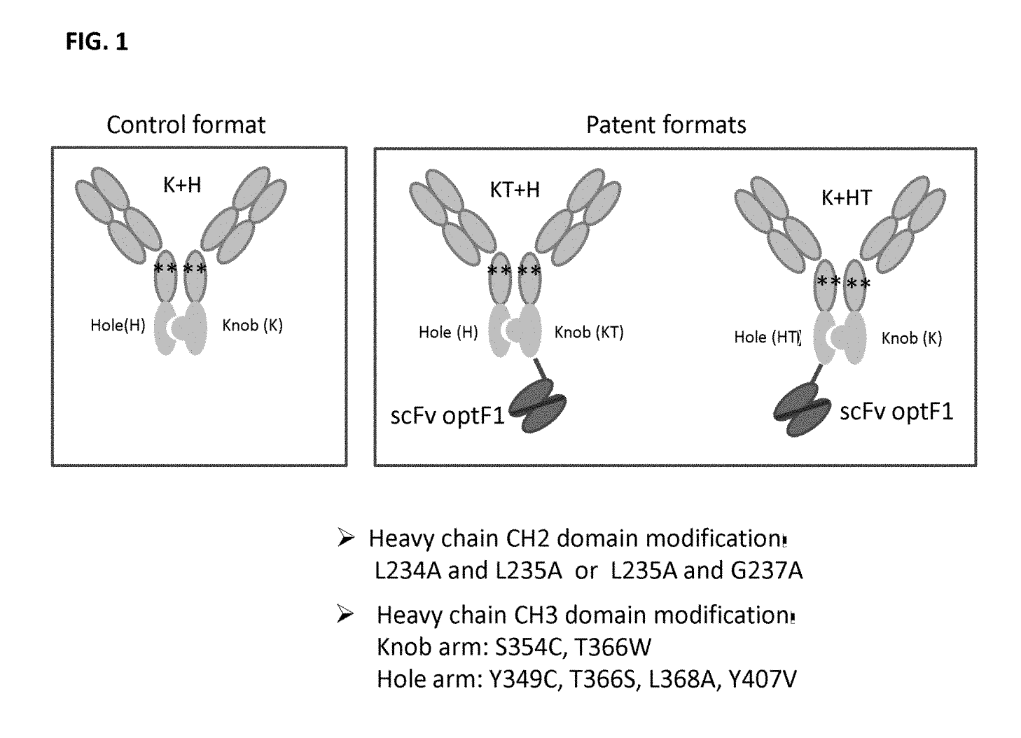

In accordance with embodiments of the invention, bispecific antibodies of the invention contain binding domains directed towards T-cells (ScFv optF1 in FIG. 1), while the antibody variable domains bind specifically to cells expressing Globo H (e.g., cancer cells). The two specific binding domains on the same molecule ensure that the T cells are brought to the target cells that express Globo H. If the effector function on a bispecific antibody of the invention is intact, the NK cells (via FcR on NK cells) can bind to the effector function site (located in in the hinge and CH2 domain) of Fc portion of the antibody, while the anti-CD3 domain (e.g., ScFv or Fab) binds CD3 on T-cells. When this occurs, the NK cells may mediate cytotoxicity towards the T cells. This would be counterproductive. In addition, the effector functions may produce ADCC or CDC that are less specific than the cytotoxicity induced by a bispecific antibody of the invention without the effector functions.

Several approaches have been disclosed in the prior art for reducing the effector functions, including glycan modifications, use of IgG2 or IgG4 subtype that do not interact well with the receptors on the effector cells, or mutations in the effector interaction sites (i.e., the lower hinges or CH2 domains) of the bispecific antibodies.

In accordance with embodiments of the invention, antibodies may be modified to reduce or diminish ADCC and CDC effector functions by site-directed mutagenesis at the effect binding site. In one example, amino acid residues at positions 234 and 235 in the CH2 domains of the knob arms and/or the hole arms are changed from leucine to alanine. In another example, amino acid residues at positions 235 and 237 in the CH2 domains of the knob arms and/or the hole arms are changed from leucine and glycine to alanine.

With mutated effector binding sites, such antibodies will be less likely to recruit effector cells (e.g., NK cells), and therefore such a bispecific antibody will not (or less likely to) trigger ADCC or CDC response on its own. Instead, T-cell engagement and activation are dependent on binding of antibodies of the invention to the antigens expressed on the surfaces of target cells, thereby increasing the efficiency of targeted therapy and reducing the undesired side effects.

In accordance with embodiments of the invention, asymmetric heterodimeric Fc-ScFv fusion anti-Globo H×anti-CD3 bispecific antibodies (i.e., an anti-Globo H antibody with its Fc domain linking to an anti-CD3 scFv or Fab or F(ab′)2) may be generated by molecular engineering of anti-Globo H antibody heavy chains. To create asymmetric dimers, the CH3 domains of the heavy chains may be mutated to create a knob structure or a hole structure. The complementarity of the knob and hole structures would facilitate the formation of heterodimeric antibodies. Methods for the generation of such knob and hole structures are known in the art.

In accordance with embodiments of the invention, to enhance Fc heterodimerization, the amino acid residues of knob arm's CH3 domain at positions 354 and 366 may be changed from serine and threonine to cysteine and tryptophan, respectively, and the amino acid residues of hole arm's CH3 domain at positions 349, 366, 368 and 407 may be changed from tyrosine, threonine, leucine and tyrosine to cysteine, serine, alanine and valine, respectively. (A. M. Merchant et al., “An efficient route to human bispecific IgG,” Nat. Biotechnol., 1998, 16:677-81; doi: 10.1038/nbt0798-677). While this example illustrates a knob-into-hole approach to heterodimeric antibodies, other methods known in the art may also be used without departing from the scope of the invention. (see e.g., A. Tustian et al., “Development of purification processes for fully human bispecific antibodies based upon modification of protein A binding avidity,” MAbs, 2016 May-June; 8(4): 828-838; doi: 10.1080/19420862.2016.1160192.)

In addition, a T-cell targeting domain (e.g., a ScFv or Fab fragment) may be fused to either the knob arm or the hole arm of the heavy chain at the C-terminus. Such fusion proteins can be readily generated using molecular cloning techniques known in the art, such as PCR. The T-cell targeting domain may be derived from an antibody that binds specifically to an antigen (or surface marker) of T-cells, such CD3. In accordance with embodiments of the invention, the T-cell targeting domain may be a ScFv or Fab fragment derived from an anti-CD3 antibody.

Furthermore, amino acid residues responsible for effector binding at both knob arm's and hole arm's CH2 domains may be changed to eliminate or reduce effector bindings, thereby minimizing or preventing ADCC or CDC. For example, residues at positions 234 and 235 may be changed from leucine to alanine, or amino acid residues at position 235 and 237 may be changed from leucine and glycine to alanine, to diminish ADCC, CDC and non-specific effector cell functions.

With the combination of CH2 and CH3 domain engineering, an anti-Globo H×anti-CD3 bispecific antibody (BsAb) of the invention can effectively engage and active T cells to induce cytokines, as well as perforin and granzymes, production by the immune cells in a Globo H expressing target cell-dependent manner. Therefore, the therapeutic safety window of engaging T cell to target tumor by asymmetric heterodimeric anti-Globo H×anti-CD3 BsAb will be significantly increased.

FIG. 1 shows schematics illustrating heterodimeric bispecific antibodies of the invention, as compare with a conventional knob-into-hole heterodimeric antibody. As shown in FIG. 1, in accordance with embodiments of the invention, to enhance Fc heterodimerization, the amino acid residues of the knob arm CH3 domain at positions 354 and 366 may be changed from serine and threonine to cysteine and tryptophan, respectively, and the amino acid residues of the hole arm CH3 domain at positions 349, 366, 368 and 407 are changed from tyrosine, threonine, leucine and tyrosine to cysteine, serine, alanine and valine, respectively.

Embodiments of the invention are bispecific antibodies that include asymmetric antibodies (heterodimeric antibodies) containing two different antigen-binding domains, one of which specifically targets T-cells, such as ScFv optF1 (derived from an anti-CD3 antibody) shown in FIG. 1.

Embodiments of the invention may be bispecific antibodies in the form of asymmetric heterodimeric Fc-ScFv (AHFS) or asymmetric heterodimeric Fc-Fab (AHFF) fusion antibody formats. In accordance with embodiments of the invention, the ScFv or the Fab fragments may be fused with either the knob arm and/or the hole arm of the antibodies. In FIG. 1, KT indicates that the T-cell targeting domain is tethered to the knob arm, while HT indicates that the T-cell targeting domain is tethered to the hole arm. In accordance with some embodiments of the invention, a bispecific antibody may have T-cell targeting domain on both the knob and hole arms, i.e., KT+HT.

Antibodies of the invention may be obtained with various expression constructs (vectors). The expression vectors may be transfected into any suitable cells for antibody expression, such as CHO cells, 293 cells, etc. Methods for the expressions and purifications of the antibodies are known in the art.

Embodiments of the invention will be illustrated with the following specific examples. One skilled in the art would appreciate that these examples are for illustration only and that other modifications and variations are possible without departing from the scope of the invention. Various molecular biology techniques, vectors, expression systems, protein purification, antibody-antigen binding assays, etc. are well known in the art and will not be repeated in detail.

### Examples

### Preparation of Anti-Globo H Monoclonal Antibodies

Bispecific antibodies of the invention may be generated starting from a monoclonal antibody against Globo H. In accordance with embodiments of the invention, a general method for the generation of monoclonal antibodies include obtaining a hybridoma producing a monoclonal antibody against Globo H.

Methods for the production of monoclonal antibodies are known in the art and will not be elaborated here. Briefly, mice are challenged with the antigen (Globo H) with an appropriate adjuvant. Then, the spleen cells of the immunized mice were harvested and fused with myeloma. Positive clones may be identified for their abilities to bind Globo H, using any known methods, such as ELISA.

In accordance with embodiments of the invention, sequences of the antibodies are determined and used as the basis for mutations to generate knob and hole structures, as well as mutations to reduce or silence the effector functions. Briefly, the total RNA of the hybridoma was isolated, for example using the TRIzol® reagent. Then, cDNA was synthesized from the total RNA, for example using a first strand cDNA synthesis kit (Superscript III) and an oligo (dT)₂₀ primer or an Ig-3′ constant region primer. Heavy and light chain sequences of the immunoglobulin genes were then cloned from the cDNA. The cloning may use PCR, using appropriate primers, e.g., Ig-5′ primer set (Novagen). The PCR products may be cloned directly into a suitable vector (e.g., a pJET1.2 vector) using CloneJet™ PCR Cloning Kit (Fermentas). The pJET1.2 vector contains lethal insertions and will survive the selection conditions only when the desired gene is cloned into this lethal region. This facilitates the selection of recombinant colonies. Finally, the recombinant colonies were screened for the desired clones, the DNAs of those clones were isolated and sequenced. The immunoglobulin (IG) nucleotide sequences may be analyzed at the international ImMunoGeneTics information system (IGMT) website. Analysis of the CDR sequences may be based on Kabat approach, or similar approaches.

Once mAbs are obtained, the mouse mAbs may be humanized or made into completely human antibodies. Procedures for the production of humanized antibodies and human antibodies are known in the art. In addition, the antibodies may be further optimized by site-directed mutagenesis. For example, alanine scanning may be performed to identify amino acid residues in CDRs that are critical or non-critical for antibody-antigen bindings. Further optimization of the bindings may be performed by screening mutants in the CDR sequences and/or framework regions. By performing these experiments, we have identified several anti-Globo H antibodies that can bind specifically to Globo H with high avidities.

In accordance with embodiments of the invention, an anti-Globo H Antibody may comprise a heavy-chain variable domain having three complementary regions consisting of HCDR1 (GYISSDQILN, SEQ ID NO:1), HCDR2 (RIYPVTGVTQYXHKFVG, SEQ ID NO:2, wherein X is any amino acid), and HCDR3 (GETFDS, SEQ ID NO:3), and a light-chain variable domain having three complementary regions consisting of LCDR1 (KSNQNLLX′ SGNRRYZLV, SEQ ID NO:4, wherein X′ is F, Y, or W, and Z is C, G, S or T), LCDR2 (WASDRSF, SEQ ID NO:5), and LCDR3 (QQHLDIPYT, SEQ ID NO:6).

### Mutagenesis of the CH2 and CH3 Domains

The anti-Globo H monoclonal antibody sequences are used as basis for site-directed mutagenesis using techniques known in the art, such as using PCR. The desired mutant clones can be confirmed with sequencing analysis.

As noted above, bispecific antibodies of the invention preferably are asymmetric dimers. Preferably, these asymmetric dimers are based on knob-into-hole approaches. For example, to generate a hole arm, the nucleotide sequences of a heavy chain CH2 and CH3 domains may be mutated to include L234A, L235A, Y349C, T366S, L368A, and Y407V mutations. Alternatively, the nucleotide sequences of a heavy chain CH2 and CH3 domains may be mutated to include L235A, G237A, Y349C, T366S, L368A, and Y407V mutations.

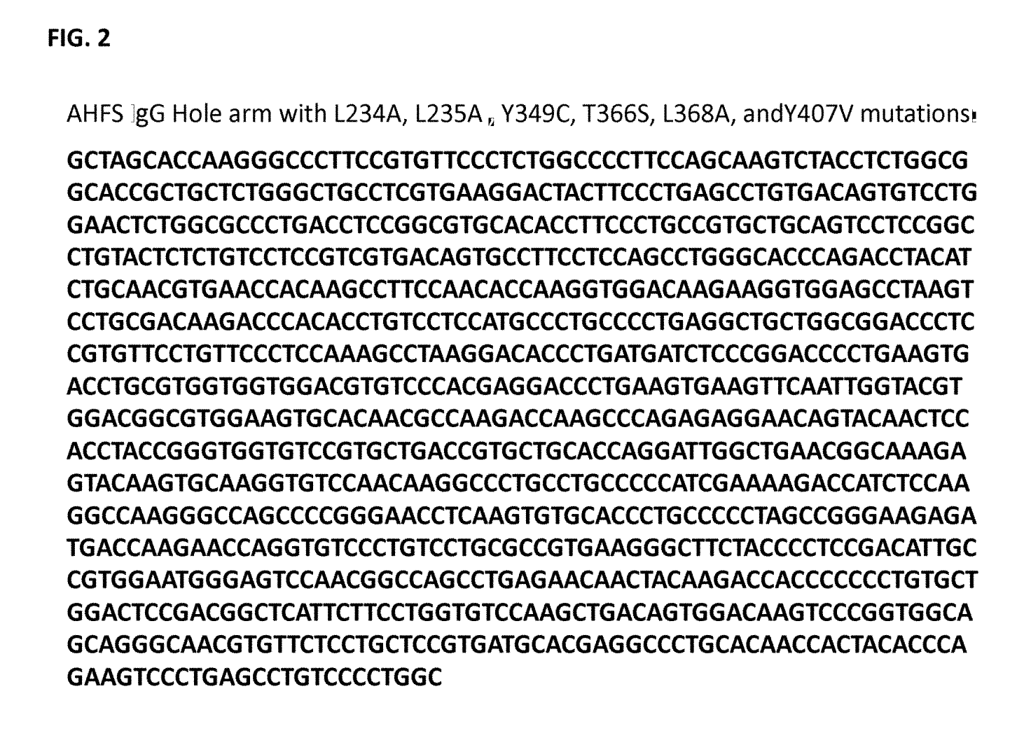

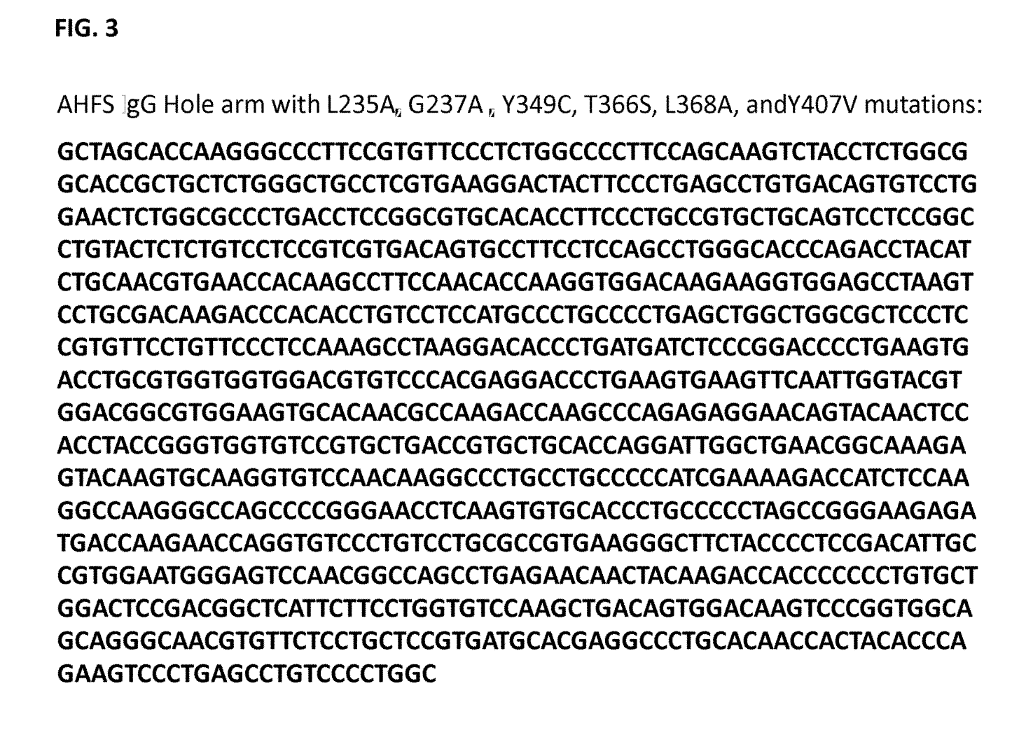

FIG. 2 shows nucleotide sequences of an exemplary hole arm with L234A, L235A, Y349C, T366S, L368A, and Y407V mutations, and FIG. 3 shows nucleotide sequences of an exemplary hole arm with L235A, G237A, Y349C, T366S, L368A, and Y407V mutations. These mutants contain mutations for the knob-into-hole mutations (residues 349, 366, 368, and 407) in the CH3 domain, as well as effector binding site mutations (residues 234, 235, and 237) in the CH2 domain. Note that these are for examples only. One skilled in the art would appreciate that similar mutations known in the art can also be used without departing from the scope of the invention.

For example, additional mutants may include the following. To generate a knob arm, the nucleotide sequence of a heavy chain CH2 and CH3 domains may be mutated to include L234A, L235A, S354C and T366W mutations. Alternatively, the nucleotide sequence of a heavy chain CH2 and CH3 domains may be mutated to include L235A, G237A, S354C and T366W mutations.

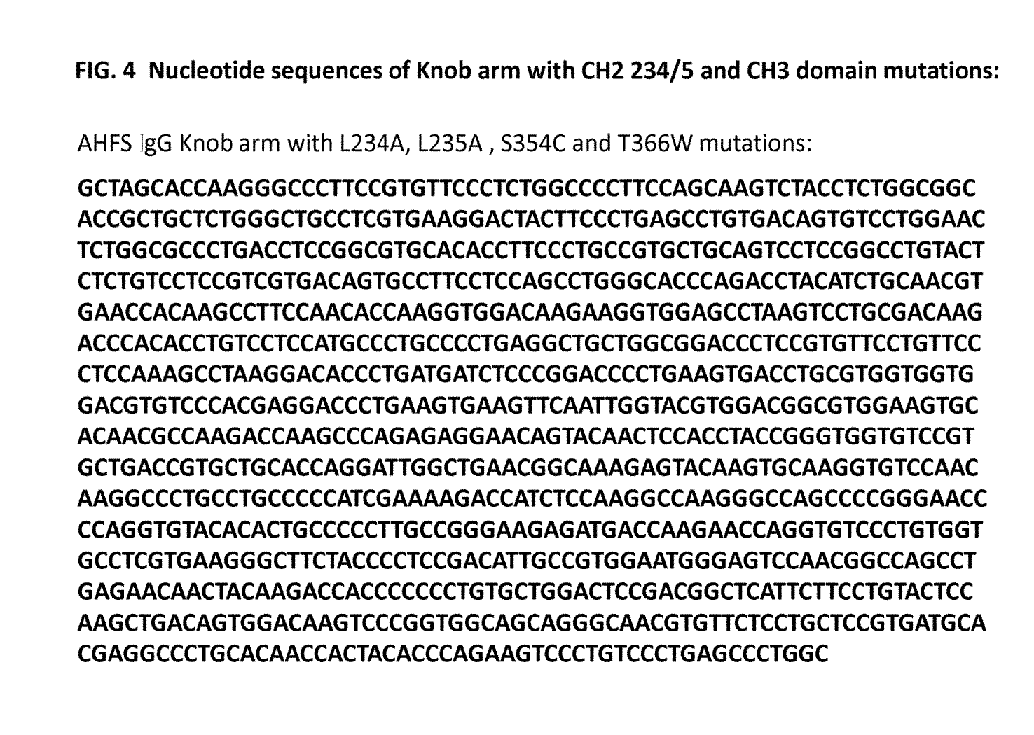

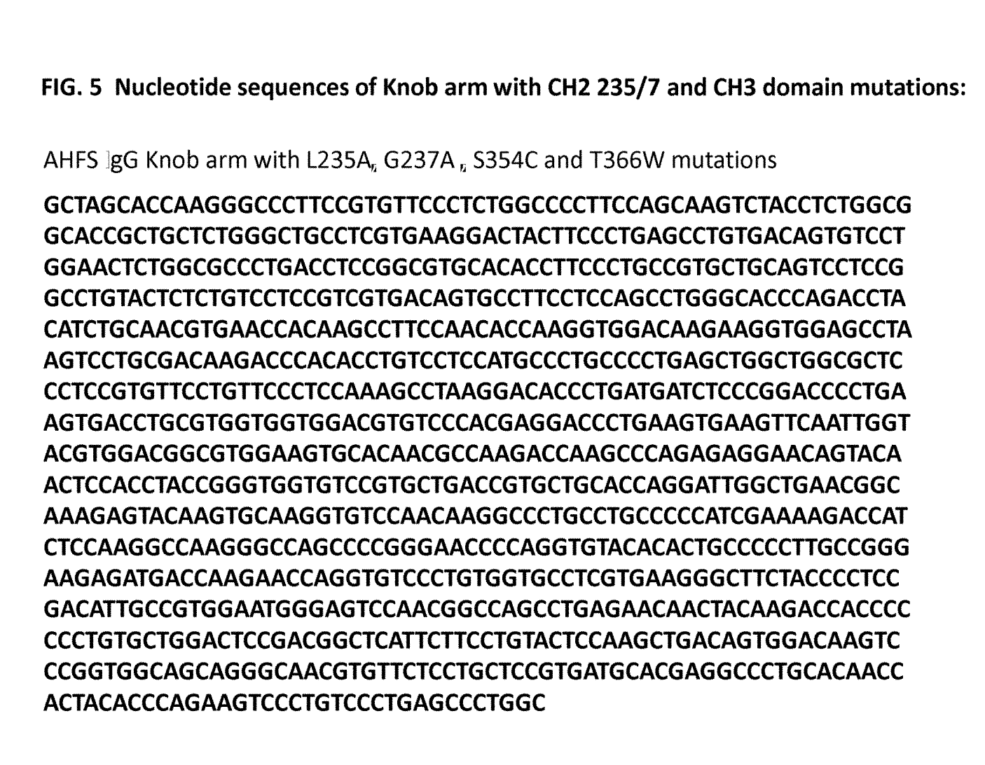

FIG. 4 shows nucleotide sequences of an exemplary knob arm with L234A, L235A, S354C and T366W mutations, and FIG. 5 shows nucleotide sequences of an exemplary knob arm with L235A, G237A, S354C and T366W mutations.

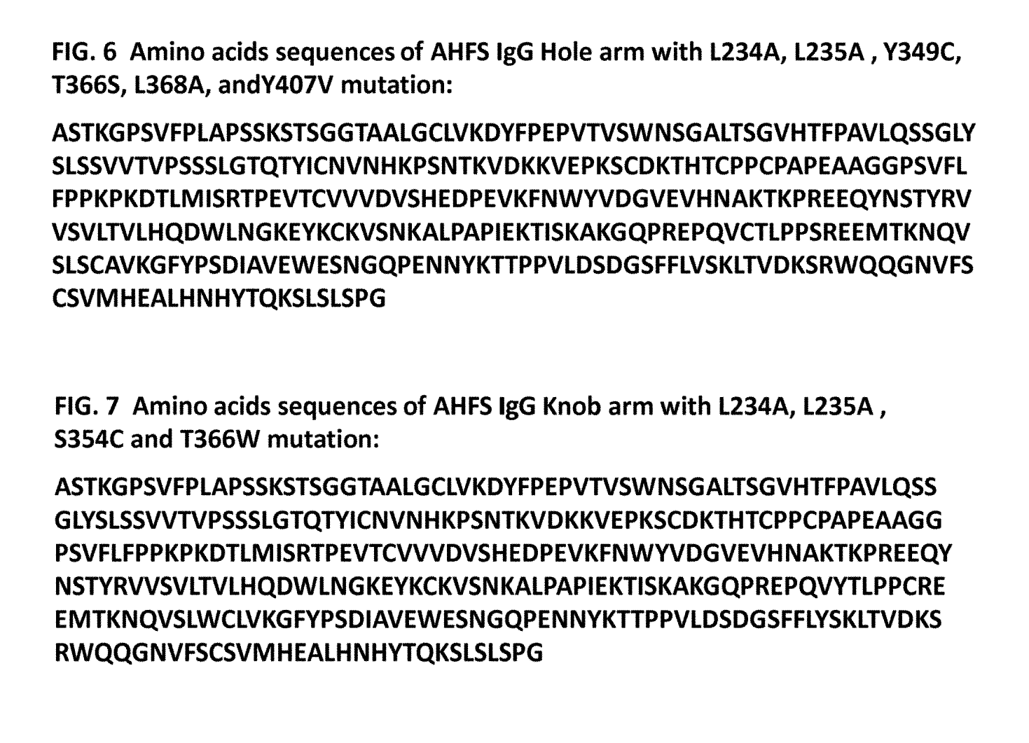

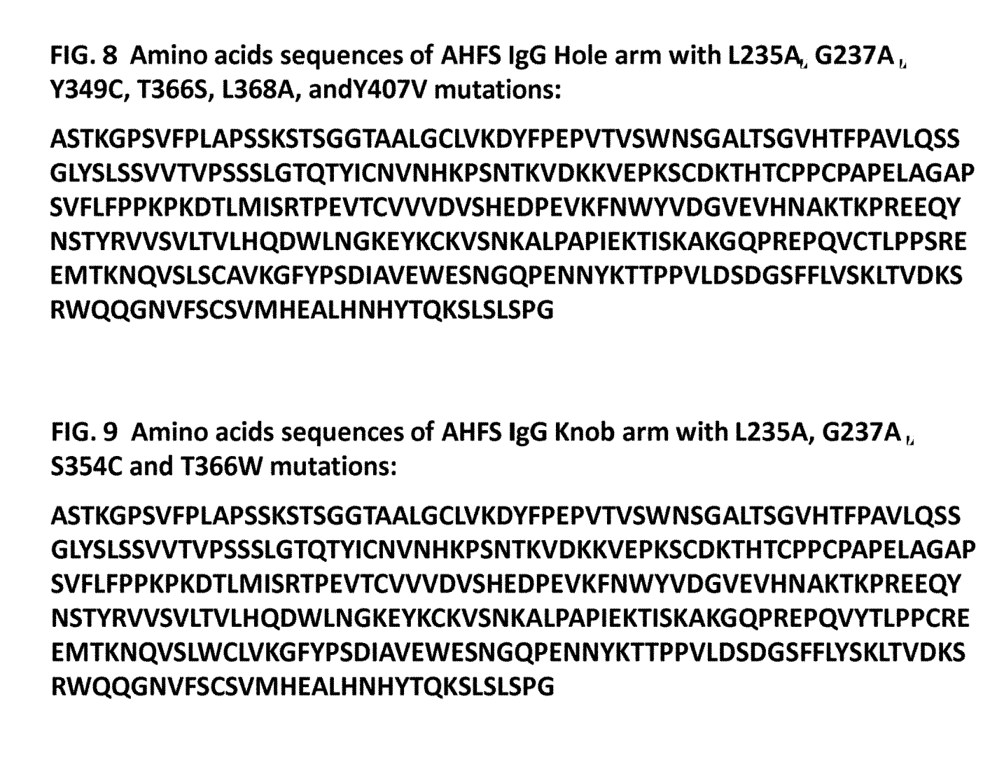

One skilled in the art would appreciate that the mutations in the CH2 and CH3 may be swapped—i.e., mix-and-match. For example, FIG. 6 shows amino acid sequences of an exemplary hole arm with L234A, L235A, Y349C, T366S, L368A, and Y407V mutations, and FIG. 7 shows amino acid sequences of an exemplary knob arm with L234A, L235A, S354C and T366W mutations. Alternatively, FIG. 8 shows the amino acid sequence of an exemplary hole arm with L235A, G237A, Y349C, T366S, L368A, and Y407V mutations, and FIG. 9 shows an amino acid sequence of an exemplary knob arm with L235A, G237A, S354C and T366W mutations.

### Generation of Bispecific Anti-Globo H×Anti-CD3 Antibodies

Bispecific antibodies of the invention each contain a T-cell targeting domain. The T-cell targeting domain, for example, may target CD3. For example, to prepare a bispecific antibody of the invention, an anti-CD3 ScFv (or Fab) may be fused to the C-terminus of an anti-Globo H antibody. A linker may be used between the anti-CD3 ScFv and the CH3 domain of the anti-Globo H antibody. Any suitable linkers may be used with embodiments of the invention, such as a short peptide linker (e.g., Gly Gly Gly Gly Ser Gly Gly Gly Gly Ser Gly Gly Gly Gly Ser; SEQ ID NO: 7).

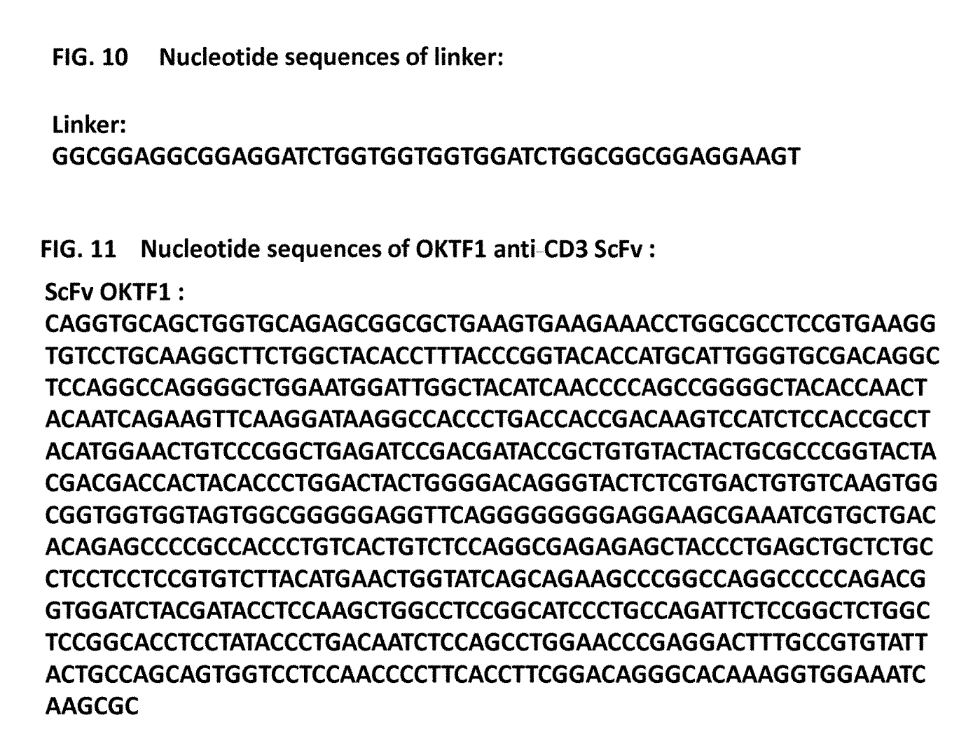

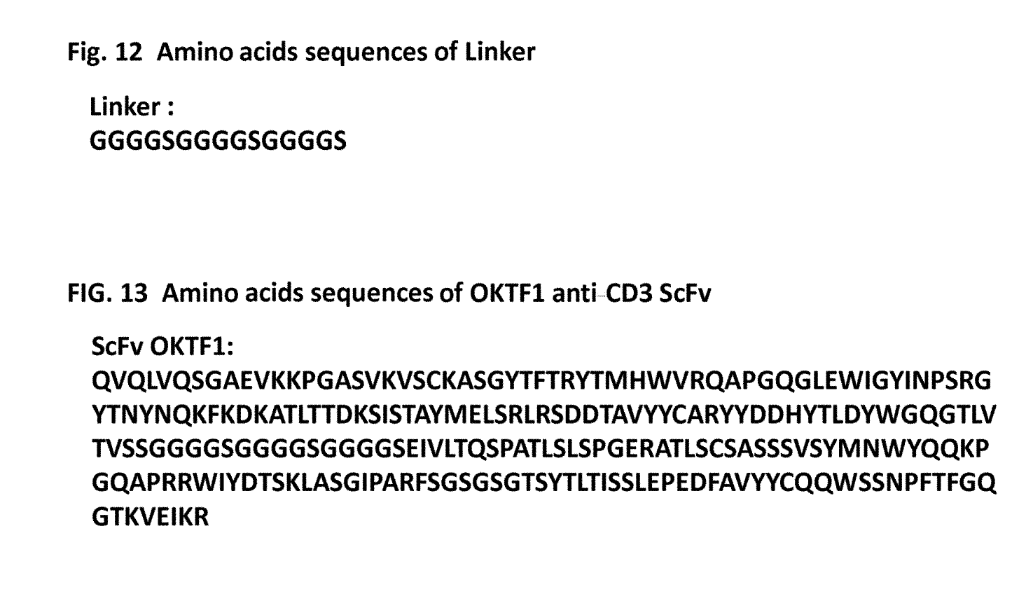

FIG. 10 shows the nucleotide sequence of one example of a linker, and FIG. 11 shows the nucleotide sequence of a ScFv of an anti-CD3 (OKTF1), while FIG. 12 and FIG. 13 show the corresponding amino acid sequences, respectively.

Generation of these antibodies requires only routine molecular biological techniques. As an example, (1) knob arm and hole arm were generated by sub-cloning of PCR amplified, synthetic knob arm gene, S354C and T366W, and hole arm gene, Y349C, T366S, L368A, and Y407V, with MfeI and BamHI digestion, into an anti-Globo H antibody expressing vector.

(2) knob arm or hole arm fused with anti-CD3 ScFv were generated by assembly PCR of synthetic knob arm-linker or hole arm-liker gene fragment with a linker-anti-CD3 ScFv gene fragment, and the assembled DNA, following MfeI and BamHI digestions, were subcloned into anti-Globo H antibody expressing vector.

(3) mutation of CH2 domain was generated, for example, by assembly PCR of synthetic gene fragment with 234A and 235A mutation or 235A and 237A mutation and the assembled DNA, following NheI and MfeI digestions, were sub-cloned into anti-Globo H antibody expressing vector.

### Antibody Expression and Purification

For antibody production, various clones may be expressed in any suitable cells, such as CHO or F293 cells. As an example, F293 cells (Life technologies) were transfected with the anti-Globo H mAb expressing plasmid and cultured for 7 days. The anti-Gobo H antibody may be purified from the culture medium using a protein A affinity column (GE). Protein concentrations may be determined with a Bio-Rad protein assay kit and analyzed with 12% SDS-PAGE, using procedures known in the art or according to the manufacturer's instructions.

The various antibodies of the invention may be analyzed with techniques known in the art, such as SDS-PAGE and HPLC. For example, the solutions of bispecific antibody samples may be analyzed by using a 4-12% non-reducing and reducing SDS-PAGE gel followed by Coomassie brilliant blue staining.

### Binding Affinity

The binding affinities of antibodies of the invention may be assessed with any suitable methods known in the art, such as Biacore.

Briefly, to a flow solution of anti-Globo H was prepared for binding kinetics studies. Ligand Globo H was immobilized on CM5 chip: First, Dilute the ligand (Globo H-amine) to 6 μg/mL in immobilization buffer (10 mM sodium acetate pH 4.5). General immobilization at 25° C. using a flow rate of 5 μL/min. Reagents for immobilization are provided in the amine coupling kit. Activation: EDC/NHS 7 minutes. Immobilization: flow time 720 seconds. Deactivation: 1.0 M ethanolamine pH 8.5 for 7 minutes. This procedure should result in response bound level about 200 RU on sensor chip CM5.

Then, the single-cycle kinetics assay was performed as followed: Biacore single-cycle kinetics (SCK) method provided with the software to obtain kinetics data. Choose Run: Method. Set the parameters as followed: Data collection rate: 1 Hz, Detection mode: Dual, Temperature: 25° C., Concentration unit: nM, Buffer A: HBS-EP+ buffer. Select the Start up and change the Number of replicates to 3. Select the Startup cycle and set the parameters as followed: Type: Low sample consumption, Contact time: 150 seconds, Dissociation time: 420 seconds, Flow rate: 50 μL/min, Flow path: Both. Select the Sample cycle and set the parameters as followed: Type: Single cycle kinetics, Concentration per cycle: 5, Contact time: 150 seconds, Dissociation time: 420 seconds, Flow rate: 50 μL/min, Flow path: Both. Select the Regeneration and set the parameters as followed: Regeneration solution: 10 mM Glycine pH2.0/1.5 (v/v=1), Contact time: 45 seconds, Flow Rate: 30 μL/min, Flow path: Both. Select the Copy of the sample and set the parameters as above. Prepare samples: Dilute the analyte antibody in running buffer to 200 nM. Prepare the concentration series from the 200 nM sample: mix 200 μL of the 200 nM solution with 200 μL running buffer to get the 100 nM solution. Continue the dilution series to obtain the following: 200, 100, 50, 25 and 12.5 nM. Prepare and position samples according to Rack Positions. Make sure everything is correct according to the Prepare Run Protocol and click Start to begin the experiment. Affinity binding curve fit using predefined model (1:1 binding) provided by Biacore T100 evaluation software 2.0.

### Antibody-Mediated ADCC Assay

Any protocols for antibody-dependent cell-mediated cytotoxicity (ADCC) known in the art may be used with embodiments of the invention. For example, the effector cells, e.g., human PBMC cells, were incubated with BsAbs on Globo H overexpressing human breast carcinoma cell line HCC1428-GFP at 10:1 E/T ratio (effector to target ratio) for 72 hr. PBS was used as negative control for BsAb. Cell viability was measured by the GFP area (μm²) of cells and analyzed with Developer Toolbox 1.9.2 by IN Cell Analyzer 6000 (GE). Experiments used PBMC cells from healthy volunteers. The experimental protocols are as follows.

HCC1428-GFP cells were pre-cultured in a suitable culture medium at 37° C. in a humidified incubator atmosphere of 5% CO₂. All cell lines were subcultured for at least three passages, cells were plated in 96-well black flat bottom plates (10,000 cells/100 μl/well for all cell lines) and allowed to adhere overnight at 37° C. in a humidified atmosphere of 5% CO₂.

A solution of AHFS anti-Globo H with anti-CD3 bispecific antibody is prepared and diluted into appropriated working concentrations 24 h after cell seeding. Aliquots of the AHFS anti-Globo H×anti-CD3 solution were added to cell culture to achieve 20 nM and 100 nM and the cells incubated for 72 hours. PBMC or T-cells are used as effector cells at a ratio of 10:1 to the target cells. Cells are examined for green fluorescence at 0 hr and 72 hrs. Cell viability was measured by the GFP area (μm²) of cells.

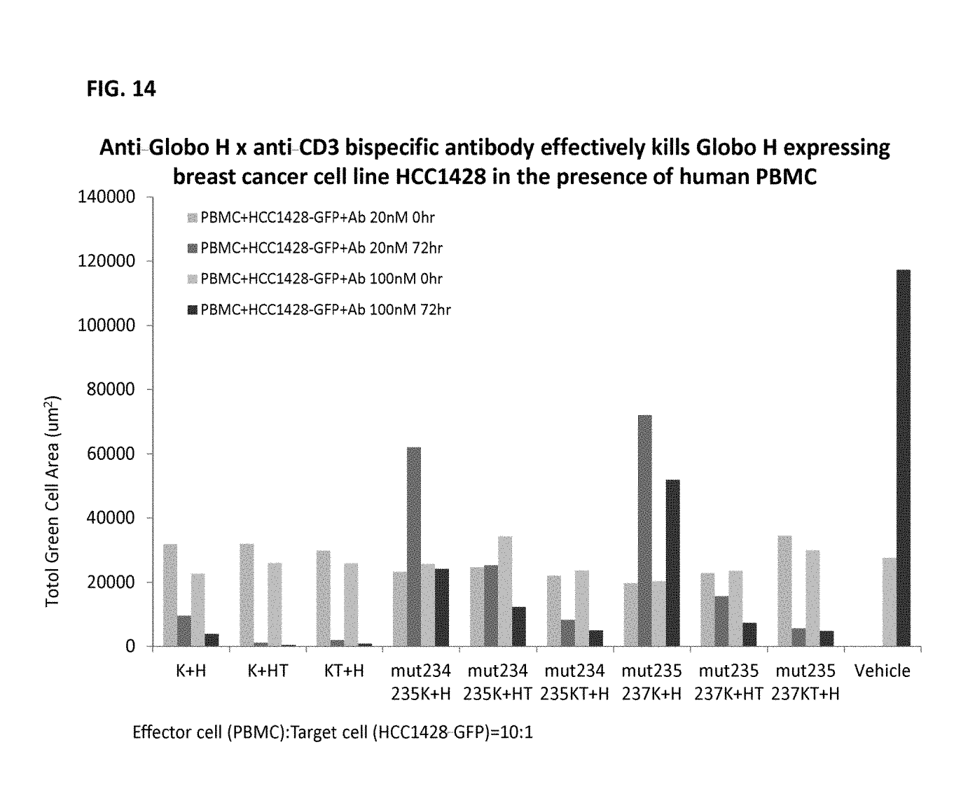

FIG. 14 shows the results of this experiment using PBMC as the effector cells. The results show that AHFS Anti-Globo H×anti-CD3 bispecific antibodies effectively kills Globo H-expressing breast cancer cell line HCC1428 in the presence of PBMC. As expected, the wild-type (i.e., without mutations to silence the effector functions) AHFS are able to kill cancer cells, with or without the anti-CD3 fusions, although the ones with anti-CD3 are more effective. These wild-type antibodies retain the ADCC and CDC functions.

In contrast, the effector-site mutants (without the effector functions) are unable to kill cancer cells without the anti-CD3 domain, indicating that the ADCC and CDC functions have been compromised. On the other hand, the antibodies with an anti-CD3 domain are able to kill cancer cells even in the absence of ADCC or CDC function. That is, with mut234-235 or mut235-237, the antibodies with tethered anti-CD3 (K+HT and KT+H) are effective in killing cancer cells, while those without (K+H) are not.

Results shown in FIG. 14 clearly show that AHFS of the invention can be engineered to have minimal or no effector functions, thereby avoiding undesired ADCC or CDC functions. However, with a T-cell targeting domain, these antibodies have the ability to kill cancer cells by T-cell specific cytotoxicity. Embodiments of the invention clearly demonstrate that bispecific antibodies against Globo H can be engineered to have no non-specific ADCC or CDC cytotoxicity and yet retain T-cell specific cytotoxicity.

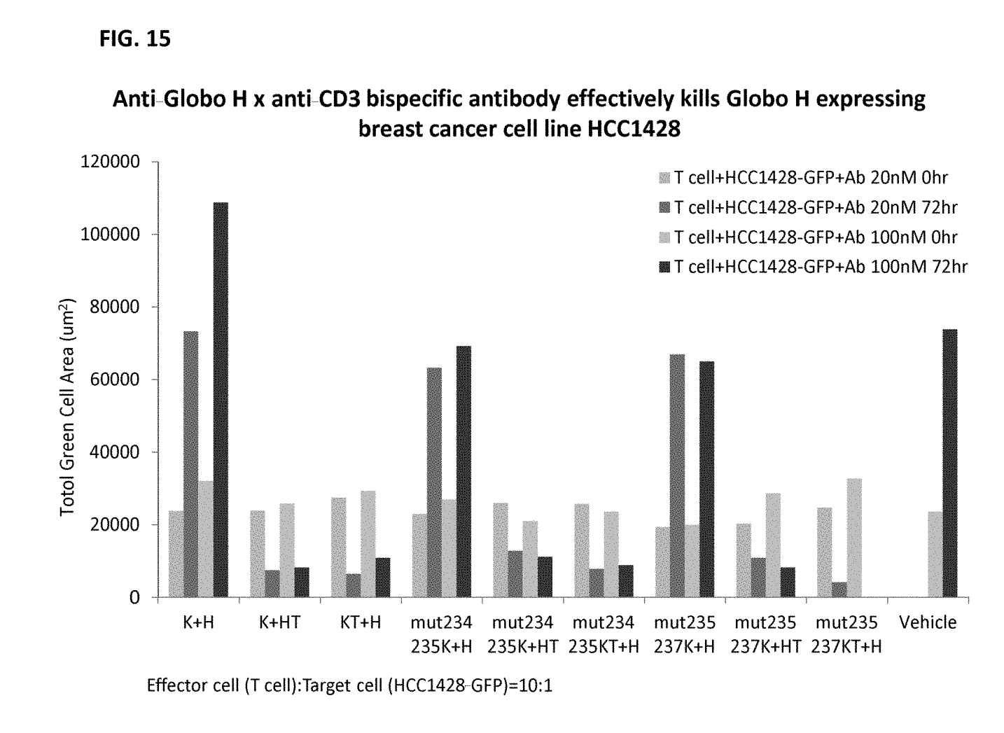

FIG. 15 shows the results of a similar experiment using T-cells as the effector cells. The results show that AHFS Anti-Globo H×anti-CD3 bispecific antibodies effectively kill Globo H-expressing breast cancer cells HCC1428. In contrast, in the absence of a T-cell targeting domain, both the wild-type (i.e., without mutations to silence the effector functions) and effector-site mutant (mut234-235 or mut235-237) AHFS are unable to engage and activate T-cells; therefore, they are unable to kill cancer cells.

Results shown in FIG. 15 clearly show that AHFS anti-Globo H×anti-CD3 bispecific antibodies of the invention can engage and activate T-cells in a specific manner, thereby avoiding non-specific ADCC.

### Antibody-Mediated CDC Assay

Any protocols for complement-dependent cytotoxicity (CDC) known in the art may be used with embodiments of the invention. For example, the complement, 40% of NHS (v:v), were incubated with bispecific antibodies (BsAbs) of the invention on Globo H overexpressing human breast carcinoma cell line HCC1428-GFP for 12 hr. PBS was used as negative control for BsAb. Detailed cell culture conditions are as outlined above. Cell viability was measured the GFP area (μm²) of cell and analyzed with Developer Toolbox 1.9.2 by IN Cell Analyzer 6000 (GE). Experiments were using NHS from health volunteer.

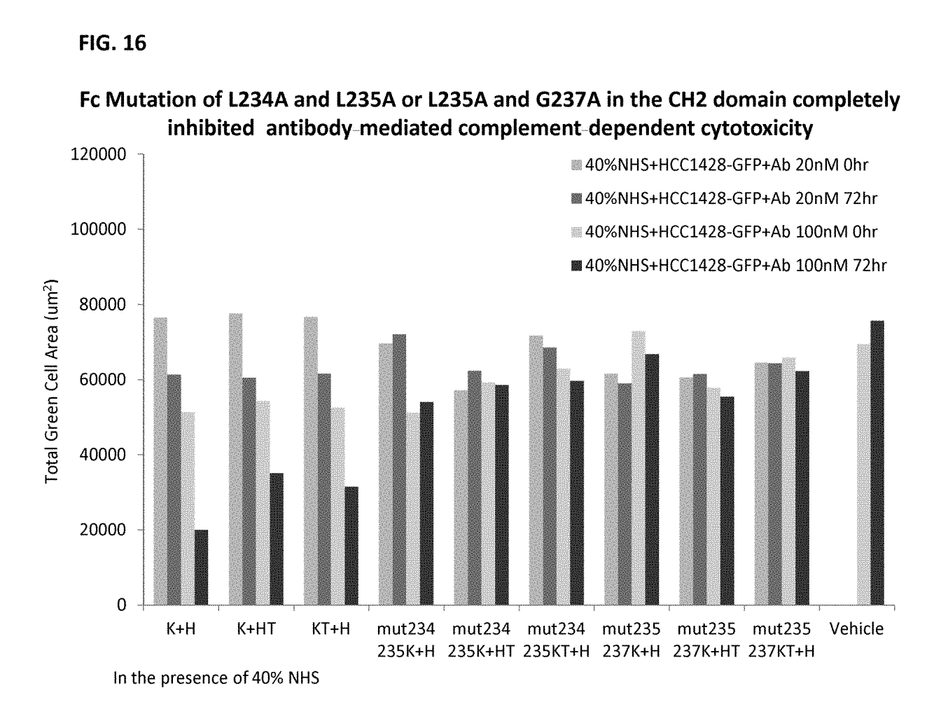

FIG. 16 shows results from this experiment. The wild-type antibodies (without mutations at the effector binding sites) are capable of supporting complement-dependent cytotoxicities (CDC). In contrast, the effector-site mutants (mut234/235 and mut235/237), which have mutations at their effector binding sites to silence the effector functions, are not able to support CDC, regardless whether the T-cell targeting domain is present or not. These results indicate that antibodies of the invention (which have mutations at their effector binding sites) will not have non-specific CDC.

### T Cell-Mediated Cytotoxicity

The above results show that antibodies of the invention with compromised effector function will not support non-specific cytotoxicities (regardless of ADCC or CDC). Instead, these antibodies support specific T-cell cytotoxicity. T-cell mediated cytotoxicity induces the production of various cytokines, as well as perforin and granzymes, which contribute to the cytotoxicity. To confirm the T-cell mediated cytotoxicity, one can assay for the productions of these factors.

The effector cells, human T cells, were incubated with BsAbs on Globo H overexpressing human breast carcinoma cell line HCC1428-GFP at 10:1 E/T ratio for 72 hr. PBS was used as negative control for BsAb. Cell viability was measured the GFP area (μm²) of cells and analyzed with Developer Toolbox 1.9.2 by IN Cell Analyzer 6000 (GE). Experiments were using T cells from health volunteer.

### Cytokine Assay

The effector cells, human PBMC cells or T cells, were incubated with BsAbs on Globo H overexpressing human breast carcinoma cell line HCC1428-GFP at 10:1 E/T ratio for 24, 48, and 72 hr. Collect supernatant and centrifuge at 800 rpm for 5 min. Supernatants were measured by Milliplex MAP Human CD8+ T Cell Magnetic Beads Panel, 6-plex plate for six cytokines (IFNγ, Granzyme A, Granzyme B, TNF-α, IL-2 and Perforin).

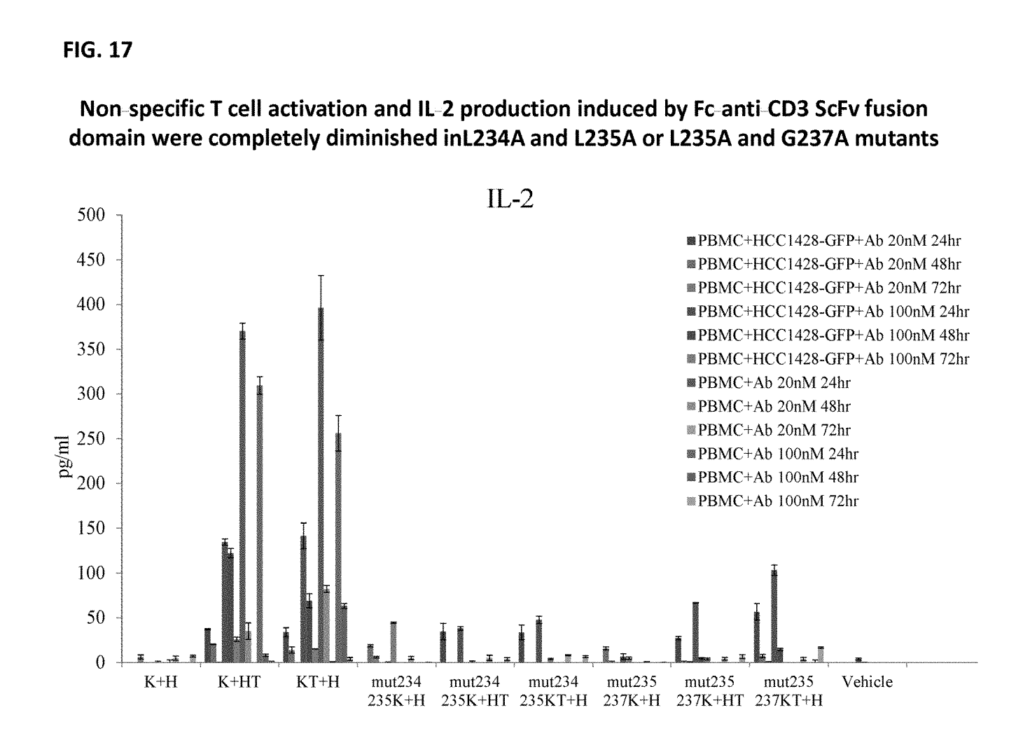

FIG. 17 shows that antibodies of the invention with mutations at the effector binding sites are not able to induce non-specific T-cell activation and IL-2 production in the absence of binding to Globo H antigen, whereas the wild-types (without mutations at the effector binding sites) can induce IL-2 production.

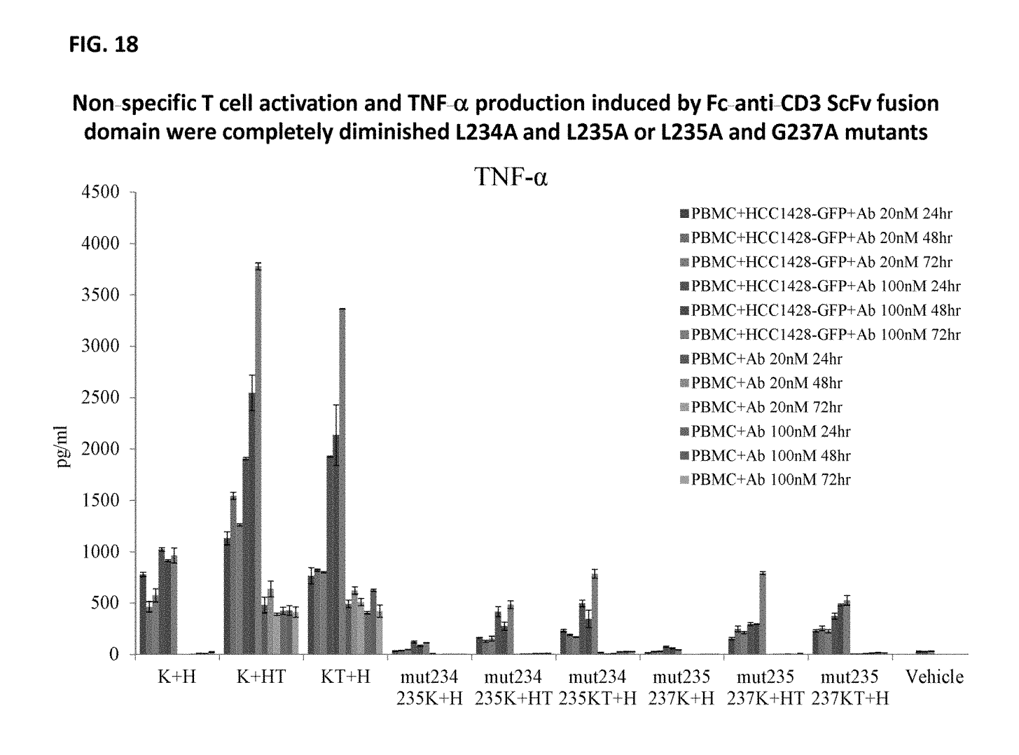

FIG. 18 shows that antibodies of the invention with mutations at the effector binding sites are not able to induce non-specific T-cell activation and TNF-α production in the absence of binding to Globo H antigen, whereas the wild-types (without mutations at the effector binding sites) can induce TNF-α production.

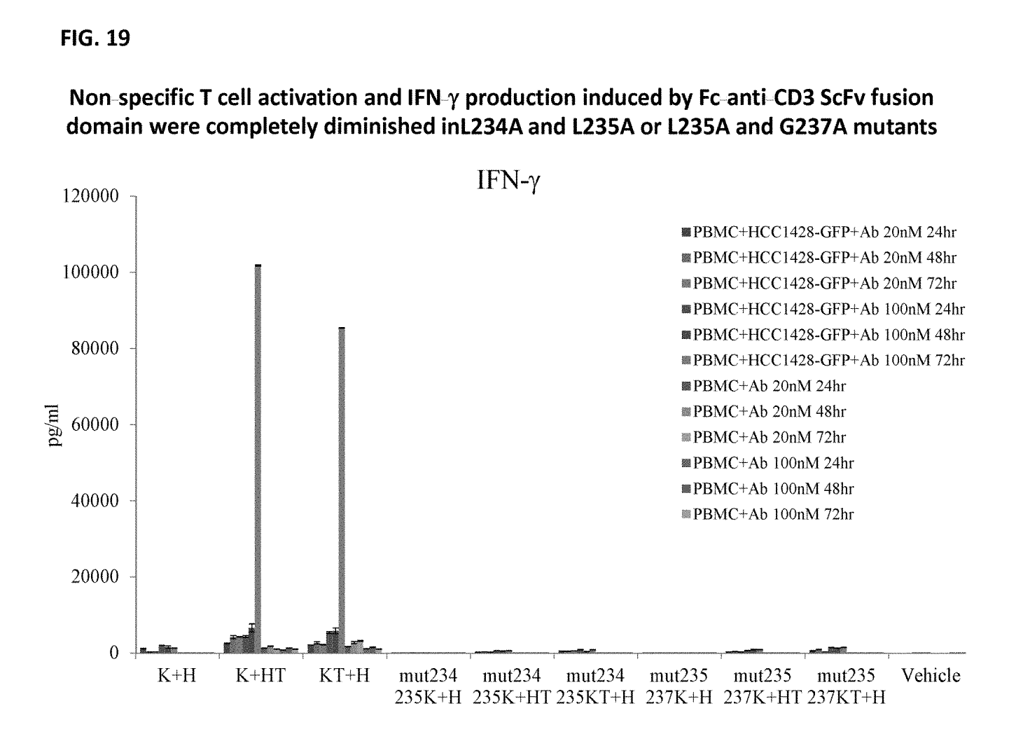

FIG. 19 shows that antibodies of the invention with mutations at the effector binding sites are not able to induce non-specific T-cell activation and INF-γ production in the absence of binding to Globo H antigen, whereas the wild-types (without mutations at the effector binding sites) can induce INF-γ production.

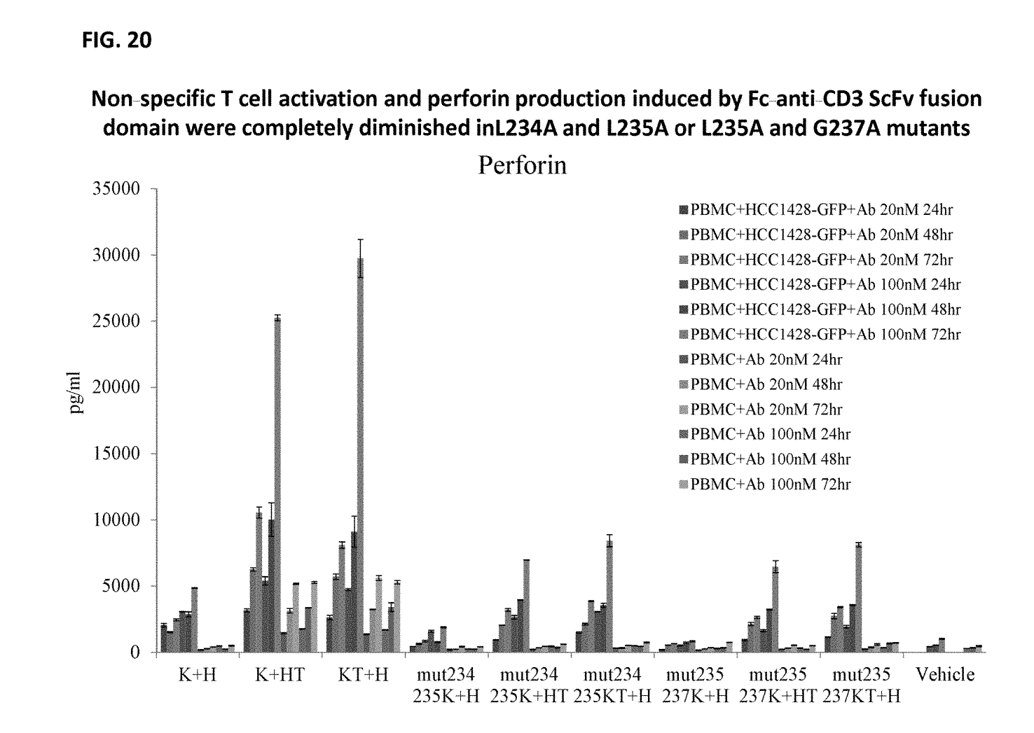

FIG. 20 shows that antibodies of the invention with mutations at the effector binding sites are not able to induce non-specific T-cell activation and perforin production in the absence of binding to Globo H antigen, whereas the wild-types (without mutations at the effector binding sites) can induce perforin production.

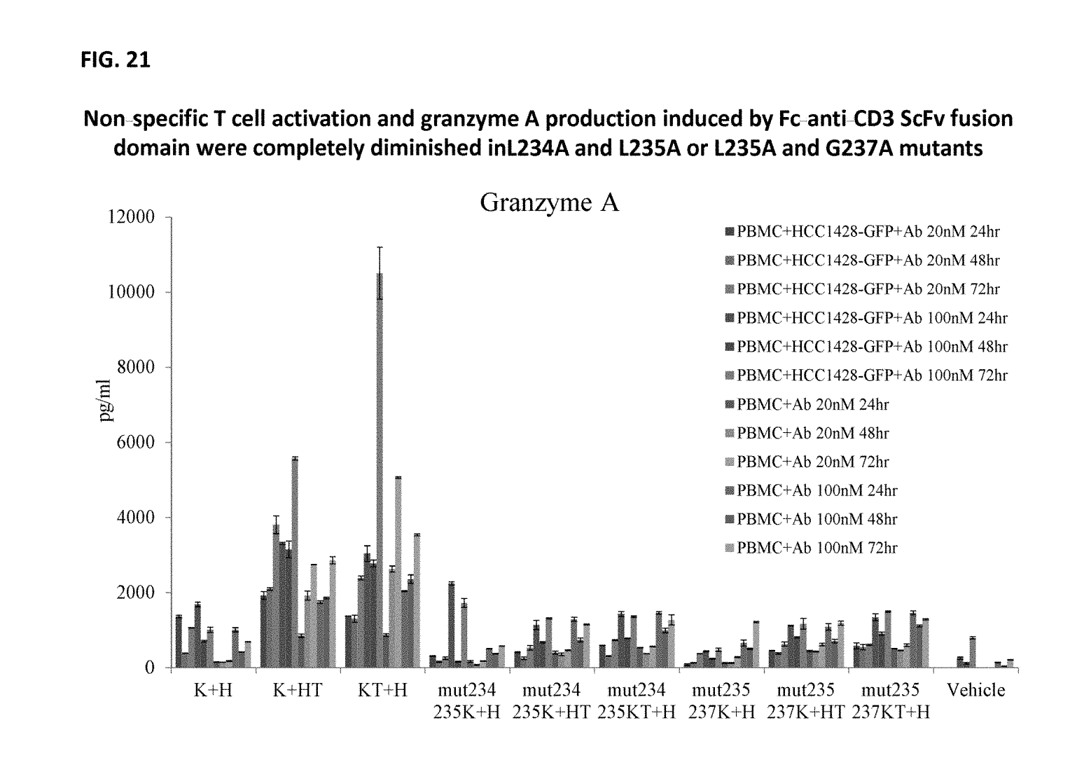

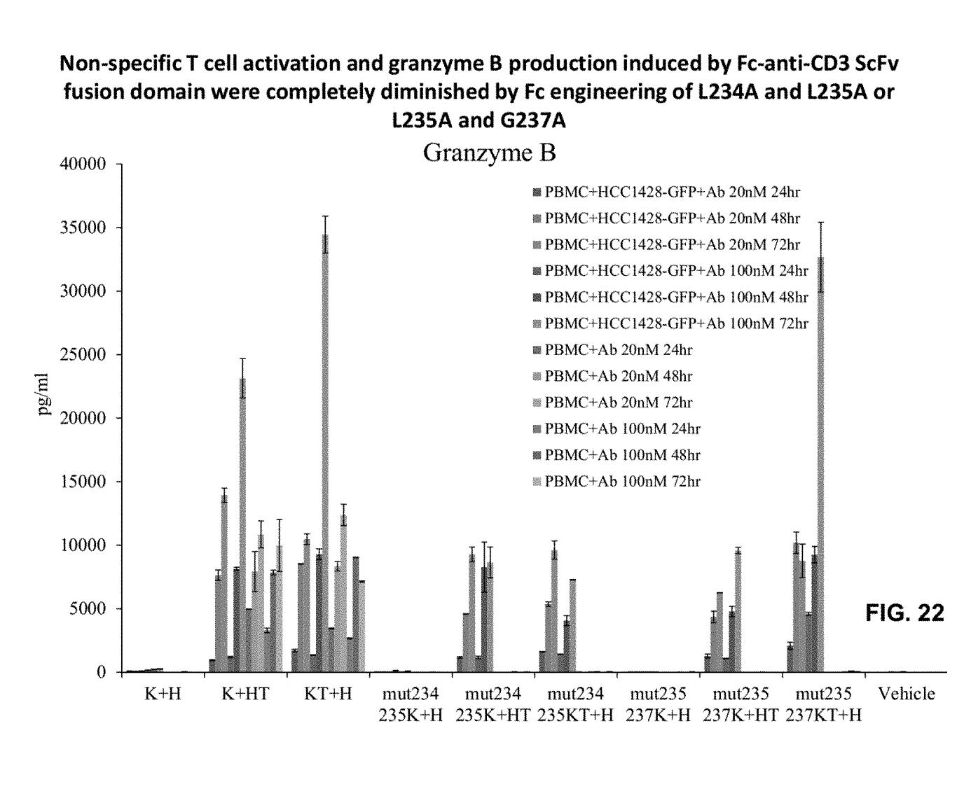

FIG. 21 shows that antibodies of the invention with mutations at the effector binding sites are not able to induce non-specific T-cell activation and granzyme A production in the absence of binding to Globo H antigen, whereas the wild-types (without mutations at the effector binding sites) can induce granzyme A production.

FIG. 22 shows that antibodies of the invention with mutations at the effector binding sites are not able to induce non-specific T-cell activation and granzyme B production in the absence of binding to Globo H antigen, whereas the wild-types (without mutations at the effector binding sites) can induce granzyme B production.

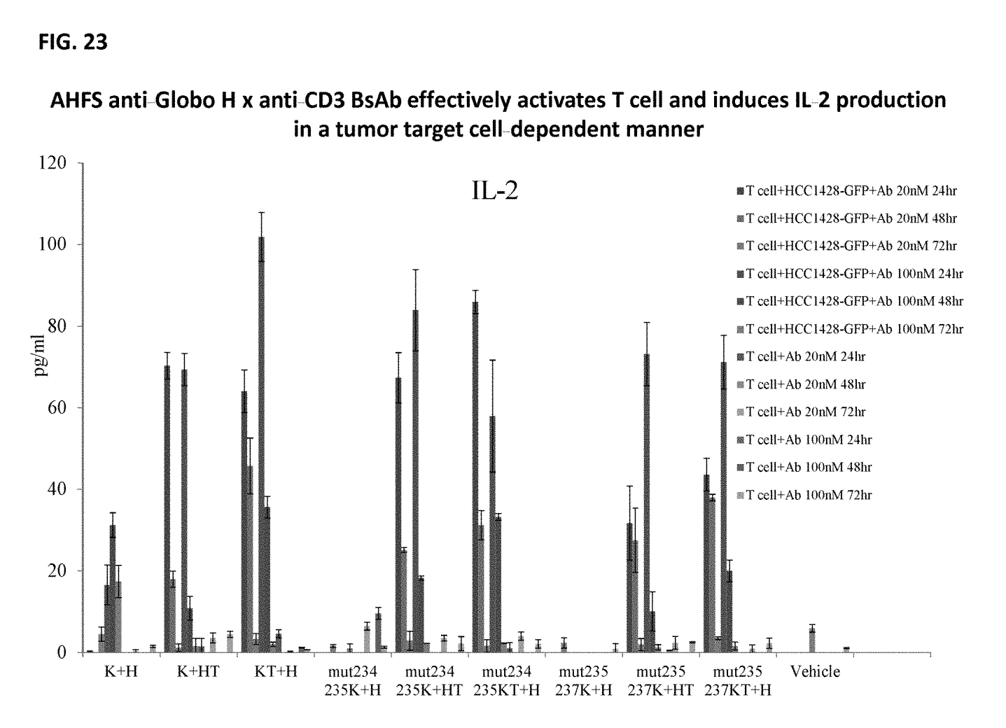

FIG. 23 shows that after binding to Globo H antigen on tumor cells, antibodies of the invention with mutations at the effector binding sites are able to engage and activate T-cells and induce IL-2 production. The specific T-cell cytotoxicity and IL-2 production induced by antibodies of the invention in the presence of Globo H antigen are almost as powerful as those induced by the wild-types (without mutations at the effector binding sites). These results indicate that the cytotoxicity induced by antibodies of the invention are tumor target cell dependent.

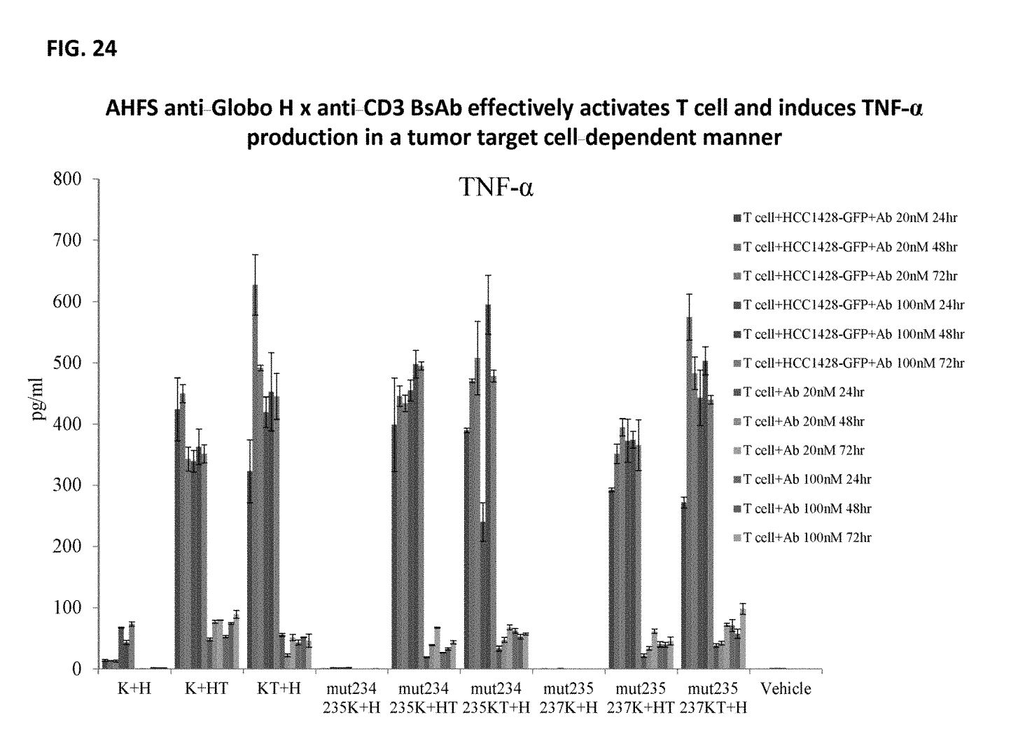

FIG. 24 shows that after binding to Globo H antigen on tumor cells, antibodies of the invention with mutations at the effector binding sites are able to engage and activate T-cells and induce TNF-α production. The specific T-cell cytotoxicity and TNF-α production induced by antibodies of the invention in the presence of Globo H antigen are almost as powerful as those induced by the wild-types (without mutations at the effector binding sites). These results indicate that the cytotoxicity induced by antibodies of the invention are tumor target cell dependent.

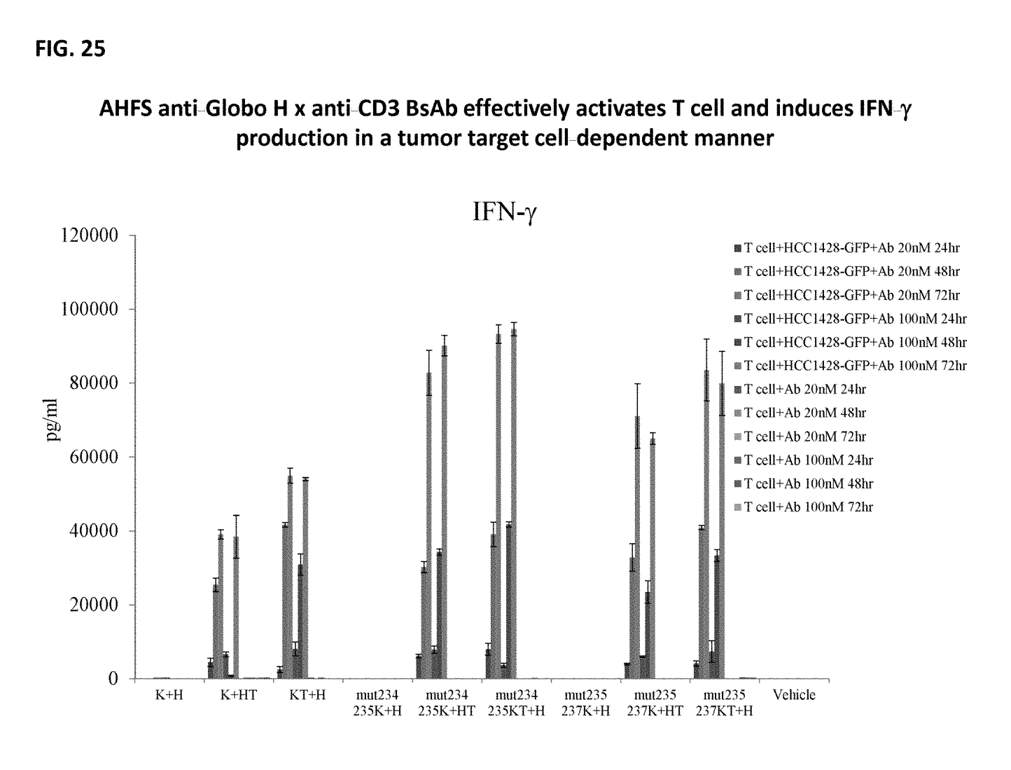

FIG. 25 shows that after binding to Globo H antigen on tumor cells, antibodies of the invention with mutations at the effector binding sites are able to engage and activate T-cells and induce INF-γ production. The specific T-cell cytotoxicity and INF-γ production induced by antibodies of the invention in the presence of Globo H antigen are almost as powerful as those induced by the wild-types (without mutations at the effector binding sites). These results indicate that the cytotoxicity induced by antibodies of the invention is tumor target cell dependent.

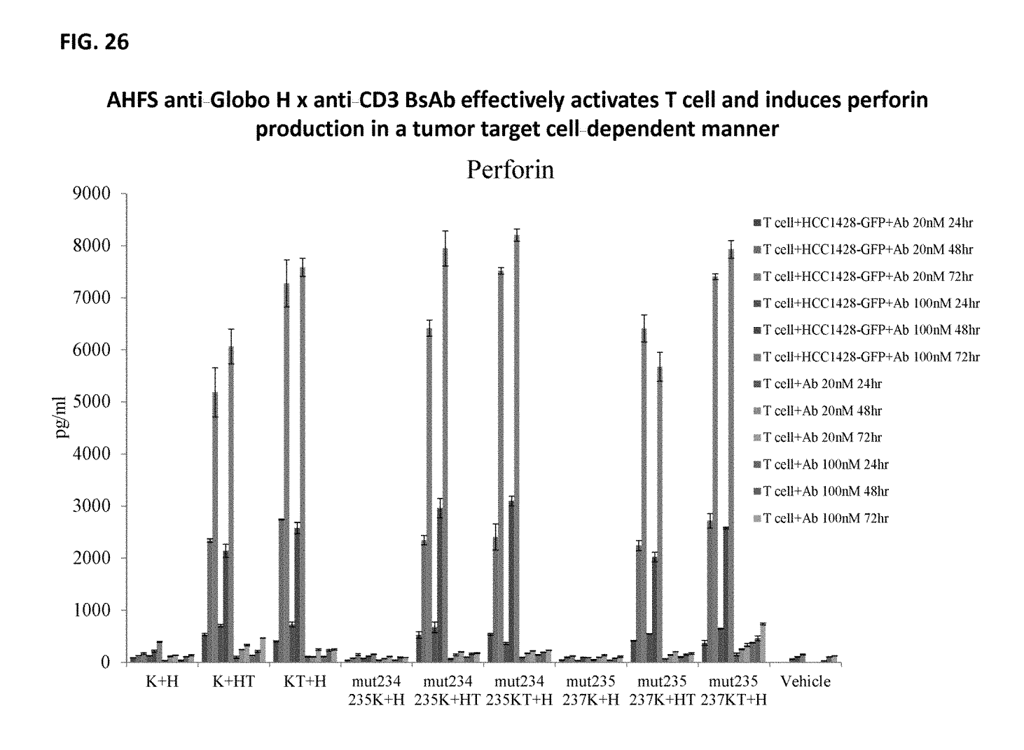

FIG. 26 shows that after binding to Globo H antigen on tumor cells, antibodies of the invention with mutations at the effector binding sites are able to engage and activate T-cells and induce perforin production. The specific T-cell cytotoxicity and perforin production induced by antibodies of the invention in the presence of Globo H antigen are almost as powerful as those induced by the wild-types (without mutations at the effector binding sites). These results indicate that the cytotoxicity induced by antibodies of the invention are tumor target cell dependent.

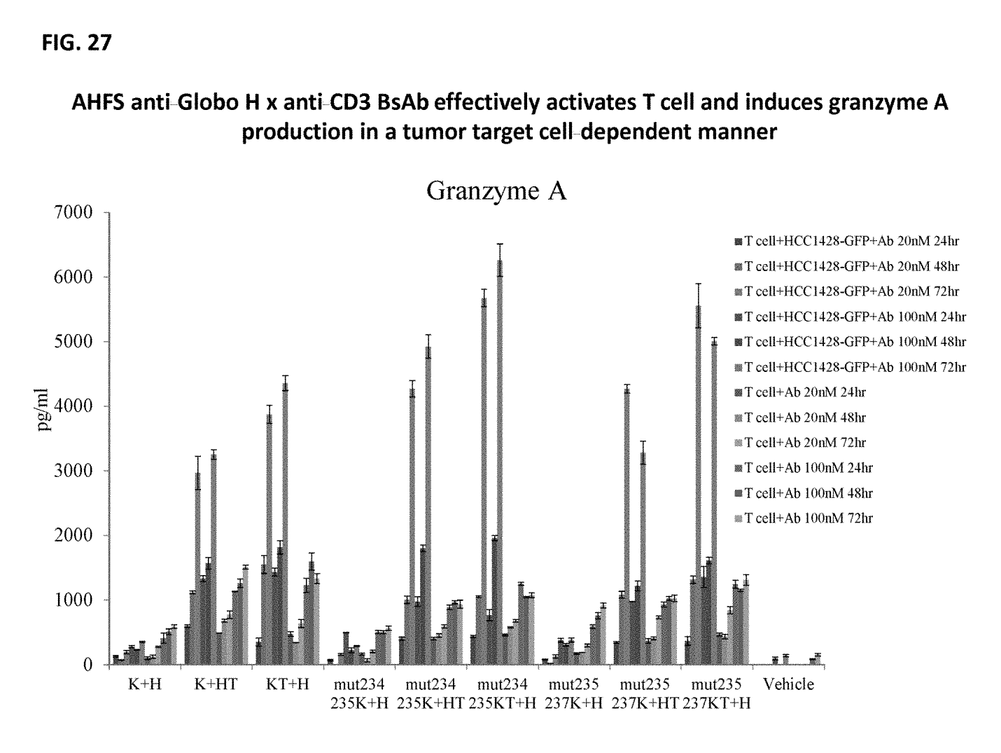

FIG. 27 shows that after binding to Globo H antigen on tumor cells, antibodies of the invention with mutations at the effector binding sites are able to engage and activate T-cells and induce granzyme A production. The specific T-cell cytotoxicity and granzyme A production induced by antibodies of the invention in the presence of Globo H antigen are almost as powerful as those induced by the wild-types (without mutations at the effector binding sites). These results indicate that the cytotoxicity induced by antibodies of the invention are tumor target cell dependent.

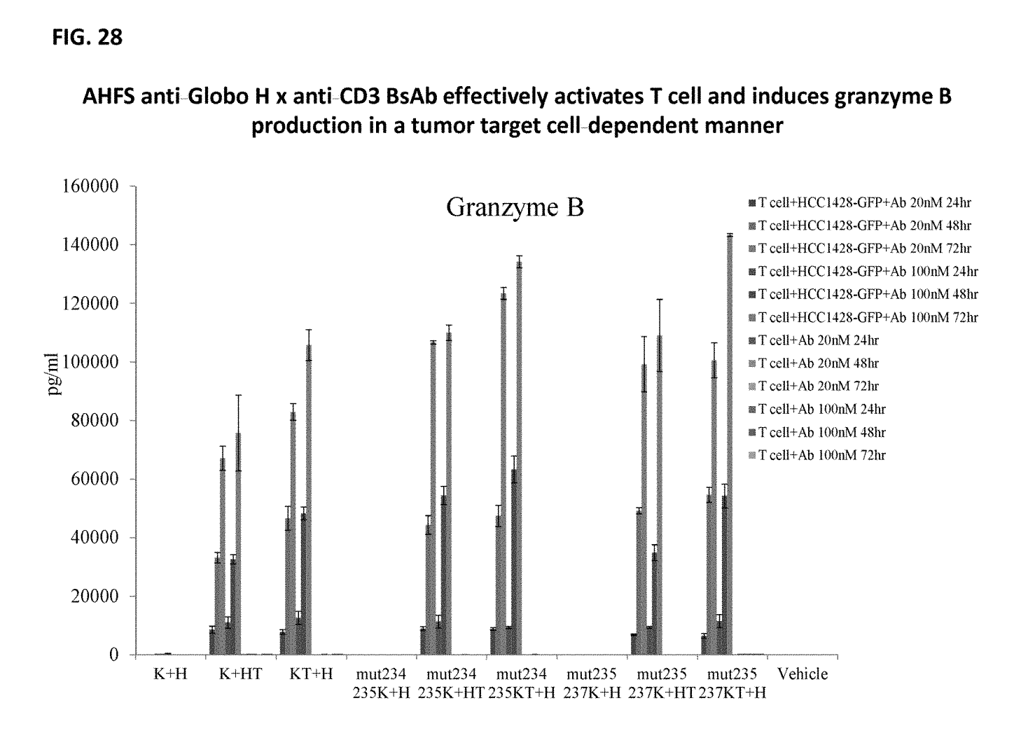

FIG. 28 shows that after binding to Globo H antigen on tumor cells, antibodies of the invention with mutations at the effector binding sites are able to engage and activate T-cells and induce granzyme B production. The specific T-cell cytotoxicity and granzyme B production induced by antibodies of the invention in the presence of Globo H antigen are almost as powerful as those induced by the wild-types (without mutations at the effector binding sites). These results indicate that the cytotoxicity induced by antibodies of the invention are tumor target cell dependent.

In sum, the above results clearly show that embodiments of the invention (anti-Globo H×anti-CD3 bispecific antibodies) with mutations at their effector binding sites will not induce non-specific ADCC or CDC. Instead, embodiments of the invention can induce specific T-cell activation (after binding to CD3 on T-cells) only in the presence of Globo H antigens, leading to the productions of (cytokines and factors that contribute to T-cells activation and tumor cell killing (e.g., IL-2, TNF-α, INF-γ, perforin, granzyme A, and granzyme B). Therefore, antibodies of the invention when used on patients will have reduced side effects and would require less antibodies to achieve the therapeutic effects.

Some embodiments of the invention relate to methods for treating cancers that are associated with expression of Globo H. Such cancers include cancers of epithelial origin, such as breast cancer, prostate cancer, lung cancer, etc. A method for treating a cancer associated with overexpression of Globo H, in accordance with embodiments of the invention, comprises administering to a subject in need thereof an effective amount of a bispecific anti-Globo H antibody as described above. The subject may be human or animals.

While embodiments of the invention have been illustrated with limited number of examples, one skilled in the art would appreciate that other modifications and variations are possible without departing from the scope of the invention. Therefore, the scope of protection of this invention should only be limited by the attached claims.

What is claimed is:

1. A bispecific anti-Globo H antibody, comprising:
an anti-Globo H antibody that binds specifically to Globo H; and
a T-cell targeting domain fused to a CH3 domain of a heavy chain of the anti-Globo H antibody,
wherein the T-cell targeting domain binds specifically to an antigen on T-cells; and
wherein the anti-Globo H antibody comprises mutations at an effector binding site such that the bispecific anti-Globo H antibody has a diminished effector function.

2. The bispecific anti-Globo H antibody according to claim 1, wherein the T-cell targeting domain is a ScFv or Fab.

3. The bispecific anti-Globo H antibody according to claim 2, wherein the ScFv or Fab is derived from an anti-CD3 antibody.

4. The bispecific anti-Globo H antibody according to claim 1, wherein the mutations at the effector binding site comprise L234A and L235A mutations.

5. The bispecific anti-Globo H antibody according to claim 1, wherein the mutations at the effector binding site comprise L235A and G237A mutations.

6. The bispecific anti-Globo H antibody according to claim 1, further comprising a knob structure in a CH3 domain of a first heavy chain and a hole structure in a CH3 domain of a second heavy chain.

7. The bispecific anti-Globo H antibody according to claim 6, wherein the knob structure is formed by S354C and T366W mutations, and the hole structure is formed by Y349C, T366S, L368A, and Y407V mutations.

8. The bispecific anti-Globo H antibody according to claim 6, wherein the anti-Globo H antibody may comprises a heavy-chain variable domain having three complementary regions consisting of HCDR1 (GYISSDQILN, SEQ ID NO:1), HCDR2 (RIYPVTGVTQYXHKFVG, SEQ ID NO:2, wherein X is any amino acid), and HCDR3 (GETFDS, SEQ ID NO:3), and a light-chain variable domain having three complementary regions consisting of LCDR1 (KSNQNLLX′SGNRRYZLV, SEQ ID NO:4, wherein X′ is F, Y, or W, and Z is C, G, S or T), LCDR2 (WASDRSF, SEQ ID NO:5), and LCDR3 (QQHLDIPYT, SEQ ID NO:6).

9. A pharmaceutical composition for treating a cancer associated with overexpression of Globo H, comprising an effective amount of the bispecific anti-Globo H antibody according to claim 1.

10. The pharmaceutical composition according to claim 9, wherein the cancer is a cancer of epithelial origin.

11. The pharmaceutical composition according to claim 10, wherein the cancer is breast cancer, colon cancer, endometrial cancer, gastric cancer, pancreatic cancer, lung cancer, or prostate cancer.
